(19) United States
(12) Patent Application Publication
(10) Pub. No.: US 2013/0003919 A1
(43) Pub. Date: Jan. 3, 2013

(54) CT Scan Device and Auxiliary Module Thereof

(76) Inventors: Chih-Kun Hsiao, Kaohsiung City (TW); Feng-Te Tsai, Kaohsiung City (TW); Yuan-Kun Tu, Kaohsiung City (TW); Ting-Sheng Lin, Kaohsiung City (TW); Li-Chang Lin, Kaohsiung City (TW); Tsung-Tien Haung, Kaohsiung City (TW)

(21) Appl. No.: 13/172,960

(22) Filed: Jun. 30, 2011

(51) Int. Cl. A61B 6/04

(52) U.S. Cl. 378/20

(57) ABSTRACT

A CT scan device including a CT scanner and an auxiliary table is proposed. The CT scanner has a scanning area and a scanning table, with the scanning table able to shift straight and pass through the scanning area. The auxiliary table is arranged on a side of the CT scanner opposite to another side where the scanning table is disposed, with the auxiliary table having an extension portion and a body, wherein a connection end of the extension portion connects with the body, an abutting end of the extension portion abutting against the scanning table, the extension portion has an inclined surface raised from the connection end to the abutting end, and the scanning table is able to push the auxiliary table moving relatively to the scanning area.

### BACKGROUND OF THE INVENTION

1. Field of the Invention

The present invention generally relates to a CT scan device and, more particularly, to a CT scan device with an auxiliary module for convenience of x-ray scanning for an upper arm of a testee.

2. Description of the Related Art

In the present time, the number of patients or testees taking computed tomography scan (CT scan) is gradually increased since CT scan devices are popularly used for its convenience and high accuracy.

Conventionally, when proceeding the CT scan, the testee or patient has to lie down on a scanning table able to returnably shift straight in a scan area of a CT scanner, so that the camera may obtain x-ray going through the body of the testee in the scan area, and thus a computer of the CT scanner may calculate and produce the cross-section of the testee through the image obtained by the camera.

However, in the operation of the above said CT scanner, in cases wherein the affected part is on the upper arm of the patient only, the body of the tested patient may be unnecessarily exposed to the x-ray. In accordance with statistical materials, 98% of a total amount of received artificial radiation dosage of a person are for medical testing, especially for CT scan, and the more the amount of radiation dosage a testee has, the higher cancer risk the testee takes.

In light of this, it is desired to improve the conventional CT scanner suitable for scanning the upper arm of a testee so as to decrease the radiation dosage taken by the testee.

### SUMMARY OF THE INVENTION

It is therefore the primary objective of this invention to provide an auxiliary module of CT scan device which can largely decrease the received artificial radiation dosage of the testee in CT scan, so as to lower the cancer risk the testee takes.

The invention discloses a CT scan device including a CT scanner and an auxiliary table. The CT scanner has a scanning area and a scanning table, with the scanning table able to shift straight and pass through the scanning area. The auxiliary table is arranged on a side of the CT scanner opposite to another side where the scanning table is disposed, with the auxiliary table having an extension portion and a body, wherein a connection end of the extension portion connects with the body, an abutting end of the extension portion abutting against the scanning table, the extension portion has an inclined surface raised from the connection end to the abutting end, and the scanning table is able to push the auxiliary table moving relatively to the scanning area.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 shows a perspective view of a CT scan device according to a preferred embodiment of the invention.

FIG. 2 shows a perspective view of an auxiliary table of the CT scan device according to the preferred embodiment of the invention.

FIG. 3 shows a side-sketch view of the CT scan device in use according to the preferred embodiment of the invention.

FIG. 4 shows another side-sketch view of the CT scan device in use according to the preferred embodiment of the invention.

In the various figures of the drawings, the same numerals designate the same or similar parts. Furthermore, when the term “first,” “second,” “bottom” and similar terms are used hereinafter, it should be understood that these terms refer only to the structure shown in the drawings as it would appear to a person viewing the drawings, and are utilized only to facilitate describing the invention.

### DETAILED DESCRIPTION OF THE INVENTION

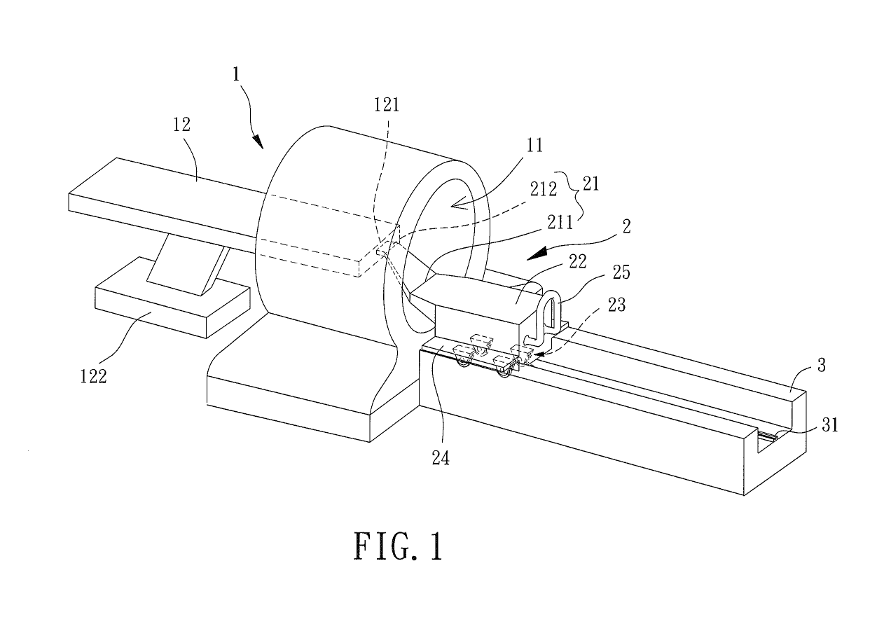

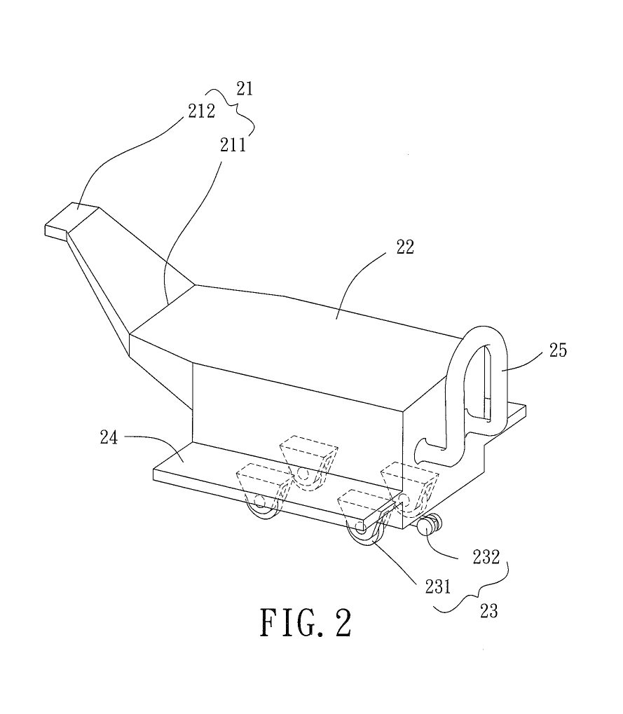

Referring to FIGS. 1 through 3, a preferable embodiment of the invention is shown, which includes a CT scanner 1 and an auxiliary table 2 detachably arranged on a side of the CT scanner 1 for a testee to lie down thereon. Hereinafter, an assembly of elements other than the CT scanner 1 is defined as an auxiliary module, no matter this assembly includes the auxiliary table 2 only or further includes a base 3 supporting the auxiliary table 2.

The CT scanner 1 is a machine able to proceed CT scan, preferably a 64-slice CT scanner, and has a scanning area 11 and a scanning table 12 able to returnably shift straight relatively to and pass through the scanning area 11. In this embodiment, a radiation emitting member 111 able to emit radioactive rays, such as the x-ray, is arranged in the scanning area 11 for proceeding the CT scan. The scanning table 12 has a first abutting end 121 adapted to abut against and push the auxiliary table 2, so as to drive the auxiliary table 2 moving along a route whereon the scanning table 12 moves. Besides, there is preferable a driving base 122 for the scanning table 12 to be mounted thereon and able to drive the scanning table 12 moving along the said route and passing through the scanning area 11 to be exposed to the radioactive ray emitted by the radiation emitting member 111. Please be noted that detailed structures of the CT scanner 1 are not further illustrated hereinafter since the CT scanner 1 is conventional.

The auxiliary table 2 is arranged on a side of the CT scanner 1, which is opposite to another side where the scanning table 12 is disposed. The auxiliary table 2 has an extension portion 21 and a body 22 for the extension portion 21 to connect with. Besides, a distal end of the extension portion 21 is able to abut against the scanning table 12 and to pass through the scanning area 11 of the CT scanner 1. The extension portion 21 can be integrally formed on the body 22 or be firmly mounted onto the body 22 after the extension portion 21 and body 22 are separately made.

In detail, the extension portion 21 has a connection end 211 and a second abutting end 212, wherein the connection end 211 connects with the body 22 and the second abutting end 212 forms the said distal end abutting against the first abutting end 121 of the scanning table 12, so that the scanning table 12 may move the auxiliary table 2 when the driving base 122 works. Preferably, the extension portion 21 has an inclined surface gradually raised from the connection end 211 to the second abutting end 212. The body 22 has a supporting surface 221 for supporting the testee, and there may be a soft pad on the supporting surface 221 of the body 22 to provide a comfortable touch. Furthermore, an angle between the inclined surface of the extension portion 21 and the supporting surface 221 is preferably adjustable for suiting all testees with different builds. There is a shifting member 23 at the bottom of the body 22 for the convenience of moving the auxiliary table 2 with low friction. Specifically, in this embodiment, the shifting member 23 includes a plurality of casters 231 and a guiding-wheel 232, with the casters 231 preferable arranged in two lines on two sides of the guiding-wheel 232.

The said base 3 supporting the auxiliary table 2 has a rail 31 extending in the direction of the said route, with the guiding-wheel 232 engaging with and able to slide along the rail 31. Preferably, the rail 31 is arranged at the center of a ditch of the base 3, which receives the auxiliary table 2, and the casters 231 are arranged in two lines on two sides of the rail 31, with two casters 231 in each line, so that the casters 231 may lower the friction between the auxiliary table 2 and the base 3 while the guiding-wheel 232 stably guides the auxiliary table 2 moving in the direction of the said route. Moreover, the guiding wheel 232 is preferably disposed at an end of the body 22 opposite to another end of the body 22 for the extension portion 21 to mount or form, so as to prevent the auxiliary table 2 from swinging when a testee lie down and lean on the extension portion 21. Instead of the casters 231 and guiding-wheel 232, the shifting member 23 can also be formed by a conventional linear guideway, which may provide a preferable lateral stability.

Specifically, the auxiliary table 2 can further provide a limiting member 24 at lateral sides of the auxiliary table 2 for stabling the position of the auxiliary table 2 in the base 3. In this embodiment, the limiting member 24 is completed by two opposite plates respectively arranged on two opposite lateral sides of the body 22, so that these two opposite plates can respectively rest on two sidewalls of the base 3, with these two sidewalls extending parallel to the rail 31 and defining the ditch. Furthermore, a handle member 25 mounted on an end of the body 22 opposite to the end connected with the extension portion 21 is provided for user to conveniently pull the auxiliary table 2 to surely abut against the scanning table 12 of the CT scanner 1. Particularly, the handle member 25 is disposed in the center of the end of the body 22 and in the form of a U-shaped pipe vertically protruding over the supporting surface 221 for the testee to hook or clip by feet.

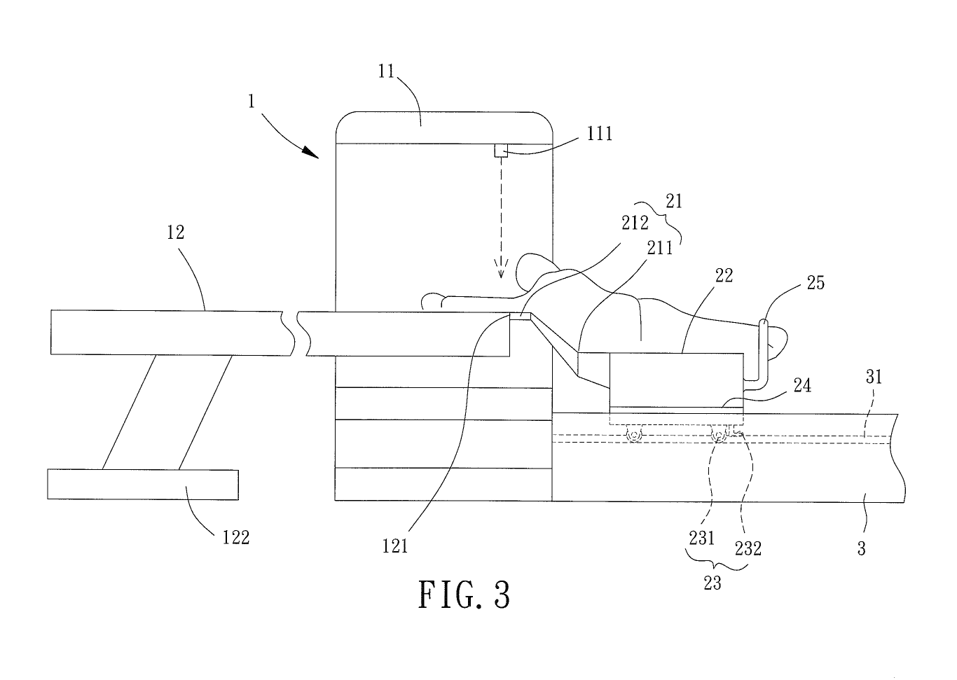

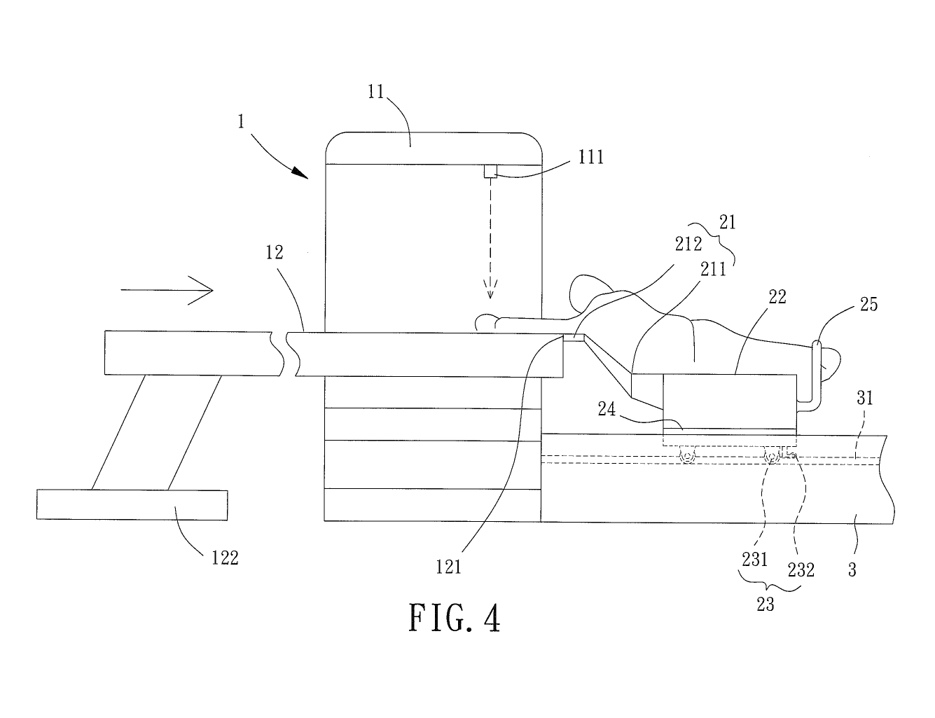

Referring FIGS. 3 and 4, when the present CT scan device is operated, the driving base 122 drives the scanning table 12 to move toward the scanning area 11 till the first abutting end 121 of the scanning table 12 connecting with the second abutting end 212 of the extension portion 21 first. Then, the testee may lie down on the supporting surface 221 of the body 22 and lean on the inclined surface of the extension portion 21 after the scanning table 12 is steady, with the testee's upper arm raising and resting on the scanning table 12, in the scanning area 11, and under the radiation emitting member 111. Finally, the driving base 122 drives the scanning table 12 to push the auxiliary table 2 and to move further, so that the whole arm of the testee may pass through the scanning area 11 for CT scan.

Specifically, with the inclined surface of the extension portion 21, the lay down testee may easily put the upper arm on the scanning table 12, with the testee's body resting on the auxiliary table 2 out of the scanning area 11 to be released from unnecessarily exposure to radioactive rays. Therefore, the present invention can largely decrease the received artificial radiation dosage of the testee in CT scan. Besides, since only the upper arm of the testee is in the scanning area 11, the scanned upper arm can be suitably placed in the center of the scanning area 11 to avoid image distortion, so that the correctness of the result image is high.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. A CT scan device, comprising:
a CT scanner having a scanning area and a scanning table, with the scanning table able to shift straight and pass through the scanning area; and
an auxiliary table arranged on a side of the CT scanner opposite to another side where the scanning table is disposed, with the auxiliary table having an extension portion and a body, wherein a connection end of the extension portion connects with the body, an abutting end of the extension portion abutting against the scanning table, the extension portion has an inclined surface raised from the connection end to the abutting end, and the scanning table is able to push the auxiliary table moving relatively to the scanning area.

2. The CT scan device as claimed in claim 1, wherein a shifting member is arranged at a bottom of the body, the shifting member comprises a plurality of casters and a guiding-wheel, the casters are arranged in two lines on two sides of the guiding-wheel, and the casters and guiding-wheel are able to move in a same direction.

3. The CT scan device as claimed in claim 2 further comprising a base with a rail, wherein the guiding-wheel engages with the rail.

4. The CT scan device as claimed in claim 3, wherein the auxiliary table further has a limiting member comprising two opposite plates respectively arranged on two opposite lateral sides of the body, these two opposite plates respectively rest on two sidewalls of the base, and these two sidewalls extend parallel to the rail.

5. The CT scan device as claimed in claim 1, wherein the auxiliary table further has a handle member mounted on an end of the body and vertically protruding over a supporting surface of the body, and the end where the handle member is mounted is opposite to another end where the extension portion connects with.

6. An auxiliary module of CT scan device, which is adapted to arranged on a side of a CT scanner, comprising:
an auxiliary table having a body and an extension portion, wherein the body has a shifting member at a bottom of the body, the extension portion has a connection end, an abutting end, and an inclined surface, with the connection end connecting with the body and the inclined surface raised from the connection end to the abutting end.

7. The auxiliary module of CT scan device as claimed in claim 6 further comprising a base with a rail.

8. The auxiliary module of CT scan device as claimed in claim 7, wherein the shifting member comprises a plurality of casters and a guiding-wheel, the casters are arranged in two lines on two sides of the guiding-wheel, the guiding-wheel engages with the rail, and the casters and guiding-wheel are able to move in a same direction.

9. The auxiliary module of CT scan device as claimed in claim 7, wherein the auxiliary table further has a limiting member comprising two opposite plates respectively arranged on two opposite lateral sides of the body, these two opposite plates respectively rest on two sidewalls of the base, and these two sidewalls extend parallel to the rail.

10. The auxiliary module of CT scan device as claimed in claim 6, wherein the auxiliary table further has a handle member mounted on an end of the body, and the end where the handle member is mounted is opposite to another end where the extension portion connects with.
